(19) United States
(12) Patent Application Publication
(10) Pub. No.: US 2018/0160792 A1
(43) Pub. Date: Jun. 14, 2018

(54) Hair Treatment Towel and Method of Making

(71) Applicant: Michele Countryman, Lynnwood, WA (US)

(72) Inventor: Michele Countryman, Lynnwood, WA (US)

(21) Appl. No.: 15/810,285

(22) Filed: Nov. 13, 2017

Related U.S. Application Data
(60) Provisional application No. 62/434,005, filed on Dec. 14, 2016.

(51) Int. Cl. A45D 37/00; A61K 8/9789; A61K 8/9794; A61K 8/67; A61K 8/85; A61K 8/73; A61Q 5/00; A61K 8/02

(52) U.S. Cl. CPC A45D 37/00; A61K 8/9789; A61K 8/9794; A61K 8/673; A61K 8/675; A61K 2800/5922; A61K 8/85; A61K 8/731; A61Q 5/002; A61K 8/0208; A61K 8/678

(57) ABSTRACT

The hair treatment towel and method of making. The hair treatment towel provides a fibrous material, such as a paper product, that is impregnated with enhancing elements that assist with the detangling and anti-frizzing of hair. The enhancing elements generally include oils and vitamins that are a safe and non-toxic. The method of impregnating a hair treatment towel includes providing the fibrous material, saturating the fibrous material with water and the enhancing elements; and drying the fibrous material in a dehydrator.

### CROSS REFERENCE TO RELATED APPLICATION

This application claims the benefit of U.S. Provisional Application No. 62/434,005 filed on Dec. 14, 2016. The above identified patent application is herein incorporated by reference in its entirety to provide continuity of disclosure.

### BACKGROUND OF THE INVENTION

### Field of the Invention

The present invention relates to a hair treatment towel and method of making. More specifically, the present invention provides a hair treatment towel comprising a fibrous material impregnated with a plurality of oils, vitamins, and other compositions for improving hair health and detangling hair. The method of making the hair treatment towel includes a process of impregnating the plurality of oils, vitamins, and other compositions with the fibrous material and dehydrating an excess moisture therefrom. Thus, allowing the hair treatment towel to release the plurality of oils, vitamins, and other compositions when engaged with wet hair.

Devices have been disclosed in the known art that relate to hair treatment towel. These include devices that have been patented and published in patent application publications. These devices in the known art have several known drawbacks. For example, one such device fails to provide the combination of oils, vitamins, and other compositions in the present proportion.

In light of the devices disclosed in the known art, it is submitted that the present invention substantially diverges in design elements from the devices in the known art and consequently it is clear that there is a need in the art for an improvement to existing hair treatment towel devices. In this regard the instant invention substantially fulfills these needs.

### SUMMARY OF THE INVENTION

In view of the foregoing disadvantages inherent in the known types of towels and towelettes now present in the art, the present invention provides a new hair treatment towel wherein the same can be utilized for providing convenience for the user when drying and detangling wet or damp hair.

It is therefore an object of the present invention to provide a new and improved hair treatment towel impregnated with plurality of oils, vitamins, and other compositions.

It is another object of the present invention to provide a method of making the hair treatment towel that releases the plurality of oils, vitamins, and other compositions when engaged with hair.

Another object of the present invention is to provide a hair treatment towel that reduces hair tangles and frizz.

Another object of the present invention is to provide a hair treatment towel that may be readily fabricated from materials that permit relative economy and are commensurate with durability.

Other objects, features and advantages of the present invention will become apparent from the following detailed description taken in conjunction with the accompanying drawings.

### BRIEF DESCRIPTIONS OF THE DRAWINGS

Although the characteristic features of this invention will be particularly pointed out in the claims, the invention itself and manner in which it may be made and used may be better understood after a review of the following description, taken in connection with the accompanying drawings wherein like numeral annotations are provided throughout.

FIG. 1 shows a perspective view of the hair treatment towel.

FIG. 2 shows a cross sectional view taking along line 2-2 of FIG. 1 of the hair treatment towel

FIG. 3 shows a flowchart of the method of making the hair treatment towel.

### DETAILED DESCRIPTION OF THE INVENTION

Reference is made herein to the attached drawings. Like reference numerals are used throughout the drawings to depict like or similar elements of the hair treatment towel and method of making. For the purposes of presenting a brief and clear description of the present invention, the preferred embodiment will be discussed as used for treating and detangling wet hair. The figures are intended for representative purposes only and should not be considered to be limiting in any respect.

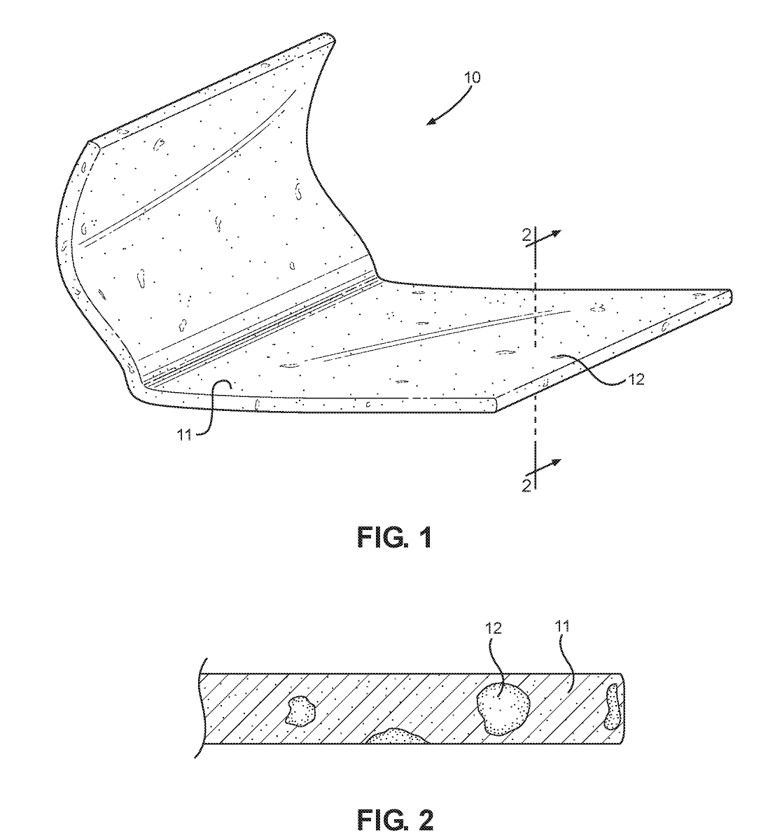

Referring to FIGS. 1 and 2, there is shown a perspective view of the hair treatment towel and a cross sectional view taking along line 2-2 of FIG. 1 of the hair treatment towel, respectively. The hair treatment towel 10 provides a fibrous material 11, such as a paper product, towel, and the like, having enhancing elements 12 impregnated therein that assist with the detangling and anti-frizzing of hair. In one embodiment, the enhancing elements 12 comprising Argan oil, Jojoba oil, and Orange oil. Each enhancing element 12 is present within the fibrous material 11 generally between 0.5%-1.5% by weight.

In the shown embodiment, the hair treatment towel 10 may further includes enhancing elements 12 of the following in any combination: Argan oil, Jojoba oil, Orange oil, Sea Buckhorn, Coconut oil, Vitamin B1, Vitamin B9, Vitamin B12, Vitamin B6, Vitamin B3, Vitamin E, Biotin, and witch hazel.

In an alternative embodiment, the hair treatment towel 10 includes a fibrous material impregnated with 0.5%-1.5% by weight of each of the following: Argan oil, Jojoba oil, Orange oil, Sea Buckhorn, Coconut oil, Vitamin B1, Vitamin B9, Vitamin B12, Vitamin B6, Vitamin B3, Vitamin E, Biotin, and witch hazel.

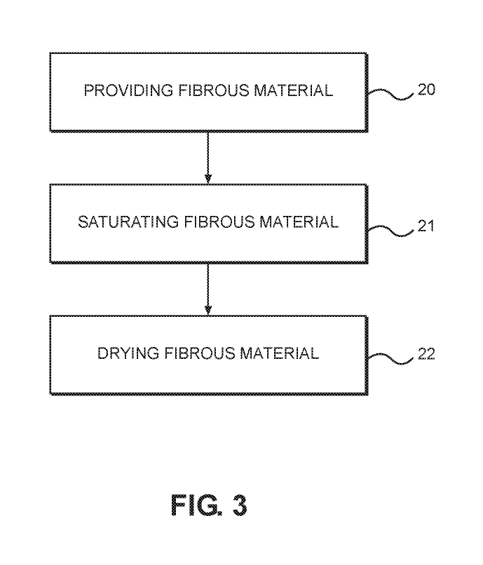

Referring to FIG. 3, there is shown a flowchart of the method of making the hair treatment towel. The method of impregnating a hair treatment towel includes: providing a fibrous material 20, saturating the fibrous material 21 with eight fluid ounces of water and a plurality of enhancing elements for at least 12 hours, the enhancing elements comprising Argan oil, Jojoba oil, and Orange oil; drying the fibrous material in a dehydrator 22, wherein the dried fibrous material is configured to release the enhancing elements. The saturation and the drying of the hair treatment towel allows for the enhancing elements to be evenly distributed throughout the hair treatment towel. In this way, upon contact with hair the enhancing elements of the hair treatment towel are released regardless of contact point with the hair. In one embodiment, each of the Argan oil, Jojoba oil, and Orange oil comprise 0.5%-1.5% by weight of the hair treatment towel.

Additionally, the method of impregnating a hair treatment towel may include: saturating the fibrous material 22 with at least one of the following enhancing elements: Sea Buckhorn, Coconut oil, Vitamin B1, Vitamin B9, Vitamin B12, Vitamin B6, Vitamin B3, Vitamin E, Biotin, and witch hazel; wherein each of the enhancing element saturating the fibrous material comprises 0.5%-1.5% by weight of the hair treatment towel. Further, a plurality of hair treatment towel may simultaneously be manufactured.

It is therefore submitted that the instant invention has been shown and described in what is considered to be the most practical and preferred embodiments. It is recognized, however, that departures may be made within the scope of the invention and that obvious modifications will occur to a person skilled in the art. With respect to the above description then, it is to be realized that the optimum dimensional relationships for the parts of the invention, to include variations in size, materials, shape, form, function and manner of operation, assembly and use, are deemed readily apparent and obvious to one skilled in the art, and all equivalent relationships to those illustrated in the drawings and described in the specification are intended to be encompassed by the present invention.

Therefore, the foregoing is considered as illustrative only of the principles of the invention. Further, since numerous modifications and changes will readily occur to those skilled in the art, it is not desired to limit the invention to the exact construction and operation shown and described, and accordingly, all suitable modifications and equivalents may be resorted to, falling within the scope of the invention.

I claim:

1) A hair treatment towel, comprising:
a fibrous material impregnated with 0.5%-1.5% Argan oil by weight, 0.5%-1.5% Jojoba oil by weight, and 0.5%-1.5% Orange oil by weight.

2) The hair treatment towel of claim 1, further comprising:
the fibrous material impregnated with 0.5%-1.5% Sea Buckhorn oil by weight.

3) The hair treatment towel of claim 1, further comprising:
the fibrous material impregnated with 0.5%-1.5% Coconut oil by weight.

4) The hair treatment towel of claim 1, further comprising:
the fibrous material impregnated with 0.5%-1.5% Vitamin B1 by weight.

5) The hair treatment towel of claim 1, further comprising:
the fibrous material impregnated with 0.5%-1.5% Vitamin B9 by weight.

6) The hair treatment towel of claim 1, further comprising:
the fibrous material impregnated with 0.5%-1.5% Vitamin B12 by weight.

7) The hair treatment towel of claim 1, further comprising:
the fibrous material impregnated with 0.5%-1.5% Vitamin B6 by weight.

8) The hair treatment towel of claim 1, further comprising:
the fibrous material impregnated with 0.5%-1.5% Vitamin B3 by weight.

9) The hair treatment towel of claim 1, further comprising:
the fibrous material impregnated with 0.5%-1.5% Vitamin E by weight.

10) The hair treatment towel of claim 1, further comprising:
the fibrous material impregnated with 0.5%-1.5% Biotin by weight.

11) The hair treatment towel of claim 1, further comprising:
the fibrous material impregnated with 0.5%-1.5% witch hazel by weight.

12) The hair treatment towel of claim 1, wherein the fibrous material comprises 70% viscose fiber and 30% polyester fiber.

13) A hair treatment towel, comprising:
a fibrous material impregnated with 0.5%-1.5% by weight of each of the following: Argan oil, Jojoba oil, Orange oil, Sea Buckhorn, Coconut oil, Vitamin B1, Vitamin B9, Vitamin B12, Vitamin B6, Vitamin B3, Vitamin E, Biotin, and witch hazel.

14) A method of impregnating a hair treatment towel, comprising;
providing a fibrous material;
saturating the fibrous material with eight fluid ounces of water and a plurality of enhancing elements for at least 12 hours, the enhancing elements comprising Argan oil, Jojoba oil, and Orange oil;
drying the fibrous material in a dehydrator, wherein the dried fibrous material is configured to release the enhancing elements.

15) The method of impregnating a hair treatment towel of claim 14, wherein each of the Argan oil, Jojoba oil, and Orange oil comprise 0.5%-1.5% by weight of the hair treatment towel.

16) The method of impregnating a hair treatment towel of claim 14, further comprising:
saturating the fibrous material with at least one of the following enhancing elements: Sea Buckhorn, Coconut oil, Vitamin B1, Vitamin B9, Vitamin B12, Vitamin B6, Vitamin B3, Vitamin E, Biotin, and witch hazel;
wherein each of the enhancing element saturating the fibrous material comprises 0.5%-1.5% by weight of the hair treatment towel.
